(19) United States
(12) Patent Application Publication
(10) Pub. No.: US 2026/0041569 A1
(43) Pub. Date: Feb. 12, 2026

(54) MEDICAL STENT AND STENT DELIVERY DEVICE

(71) Applicant: OLYMPUS MEDICAL SYSTEMS CORP., Tokyo (JP)

(72) Inventors: Keita OZAWA, Hino-shi (JP); Hirofumi ETO, Akiruno-shi (JP)

(21) Appl. No.: 18/988,349

(22) Filed: Dec. 19, 2024

(30) Foreign Application Priority Data
Aug. 7, 2024 (JP) 2024-130638

(51) Int. Cl. A61F 2/82; A61F 2/966

(52) U.S. Cl. CPC A61F 2/82; A61F 2/966; A61F 2210/0076; A61F 2220/0008; A61F 2220/0025; A61F 2230/0069; A61F 2250/0037; A61F 2250/0039

(57) ABSTRACT

What is provided are a medical stent and a stent delivery device that can be placed in a bile duct while they sufficiently conform to a shape of the bile duct.

The medical stent includes a main body including a tubular member configured to extend in a longitudinal direction, and a reinforcing material wound in a spiral shape around a longitudinal axis extending in the longitudinal direction and embedded between an inner circumferential surface of the tubular member and an outer circumferential surface of the tubular member, wherein the main body has a first region and a second region in which winding pitches of the reinforcing material are different from each other, and the second region is a region in which the reinforcing material is wound in a spiral shape at a winding pitch larger than that of the first region, and is longer than the first region in the longitudinal direction.

### BACKGROUND OF THE INVENTION

### Field of the Invention

The present invention relates to a medical stent and a stent delivery device. Priority is claimed on Japanese Patent Application No. 2024-130638, filed Aug. 7, 2024, the content of which is incorporated herein by reference.

### Description of Related Art

As a treatment for bile duct stenosis, a procedure of placing a stent in a bile duct is known. The stent placed in the bile duct is disposed along the bile duct to maintain a space within the bile duct. In the stent placed in the bile duct, a region in which the stent is placed in a part other than a stenosis portion in the bile duct needs to conform to a shape of the bile duct.

It may become necessary to replace the placed stent due to blockage, or the like. When the stent is replaced, the placed stent is first removed and retrieved.

One of methods for retrieving a stent is to retrieve the removed stent through a treatment tool channel of an endoscope.

When a stent is retrieved through a treatment tool channel, an end portion of the stent is held by a treatment tool such as grasping forceps or a snare and drawn into the channel. At this time, the end portion of the stent may bend and overlap at a part at which it is held. When the end portion of the stent overlaps, a dimension in a radial direction increases, and thus the overlapping end portion has to be collapsed in the radial direction in order to draw the stent into the channel.

Conventionally, a stent having a plurality of regions with different rigidity has been proposed (for example, Patent Document 1). In the stent having the plurality of regions with different rigidity, the region with a lower rigidity among the plurality of regions is disposed on the base end side.

When the stent is placed, the region with a greater rigidity among the plurality of regions is placed at the stenosis portion, and thus a sufficient space is maintained within the bile duct. When the stent is retrieved, a base end with the lower rigidity is collapsed to smoothly draw the stent into the channel.

### PATENT DOCUMENTS

[Patent Document 1] Japanese Patent No. 6995219

### SUMMARY OF THE INVENTION

However, in the stent placed in the bile duct, the region with the greater rigidity may have difficulty conforming to a shape of the bile duct. In particular, in the case that a length of the region with the greater rigidity is long compared to the overall length of the stent, when there is a stenosis portion in an upper portion of the bile duct, the region with the greater rigidity is disposed not only at the stenosis portion but also at parts other than the stenosis portion, which may make it difficult for the stent placed at the parts other than the stenosis portion to conform to the shape of the bile duct.

In view of the above circumstances, an object of the present invention is to provide a medical stent and a stent delivery device that can be placed in a bile duct while sufficiently conforming to a shape of the bile duct.

In order to solve the above problems, the present invention proposes the following means.

A medical stent according to the present invention includes a main body including a tubular member configured to extend in a longitudinal direction, and a reinforcing material wound in a spiral shape around a longitudinal axis extending in the longitudinal direction and embedded between an inner circumferential surface of the tubular member and an outer circumferential surface of the tubular member, wherein the main body has a first region and a second region in which winding pitches of the reinforcing material are different from each other, and the second region is a region in which the reinforcing material is wound in a spiral shape at a winding pitch larger than that of the first region, and is longer than the first region in the longitudinal direction.

A stent delivery device according to the present invention includes a guide catheter including a guide tube through which a guide wire is configured to be inserted and a wire connected to the guide tube, a pusher catheter including a first tube having a first lumen through which the wire is configured to be inserted, and a second tube having a second lumen connected to a tip end portion of the first lumen and through which the guide tube is configured to be inserted, a connecting member having a loop through which the guide tube is configured to be inserted, and connected to a tip end portion of the pusher catheter, and a stent including a main body having a tubular member configured to extend in a longitudinal direction and through which the guide tube is inserted, a reinforcing material embedded between an inner circumferential surface of the tubular member and an outer circumferential surface of the tubular member, and a through hole configured to pass through the tubular member from the inner circumferential surface to the outer circumferential surface and provided on a base end side in the longitudinal direction, the stent being located on a tip end side in the longitudinal direction with respect to a tip end of the second tube, wherein the reinforcing material is wound in a spiral shape around a longitudinal axis extending in the longitudinal direction and extends in the longitudinal direction, the main body has a first region and a second region in which winding pitches of the reinforcing material are different from each other, the second region is a region in which the reinforcing material is wound in a spiral shape at a winding pitch larger than that in the first region, and the second region is provided on a base end side with respect to the first region and is longer than the first region in the longitudinal direction, the loop of the connecting member is inserted through the through hole, and the stent is positioned with respect to the pusher catheter by inserting the guide tube through the loop.

According to the medical stent and stent delivery device of the present invention, it is possible to provide a medical stent and a stent delivery device that can be placed in the bile duct while sufficiently conforming to the shape of the bile duct.

### BRIEF DESCRIPTION OF THE DRAWINGS

FIG. 1 is an overall view of a stent delivery device according to a first embodiment.

FIG. 2 is a side view of a stent according to the first embodiment.

FIG. 3 is a perspective view of the stent.

FIG. 4 is a schematic cross-sectional view of the stent delivery device.

FIG. 5 is a diagram showing a process of placing a stent using the stent delivery device.

FIG. 6 is a diagram showing the placed stent.

FIG. 7 is a diagram showing a process of drawing the stent into a channel of an endoscope.

FIG. 8 is a perspective view of a stent according to a second embodiment.

FIG. 9 is a perspective view of a stent according to a third embodiment.

### DETAILED DESCRIPTION OF THE INVENTION

### First Embodiment

A stent delivery device 1 and a medical stent 10 according to a first embodiment of the present invention will be described with reference to the drawings.

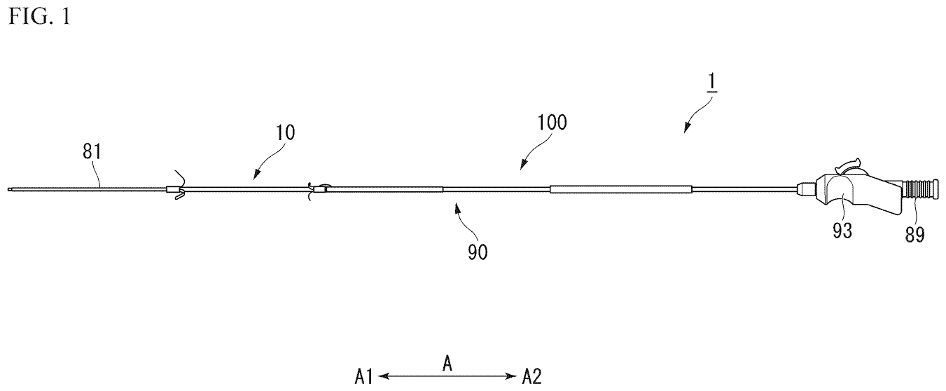

FIG. 1 is an overall view of the stent delivery device 1 according to the first embodiment.

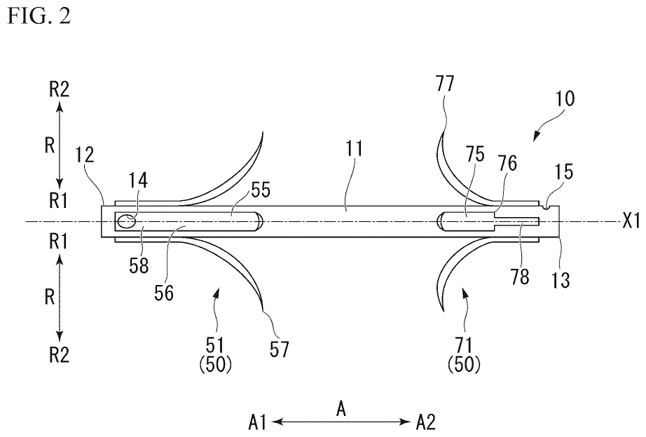

FIG. 2 is a side view of a stent 10.

The stent delivery device 1 includes a medical stent (the stent) 10 and a delivery catheter 100.

The stent 10 is a stent to be placed in a bile duct, and includes a tubular main body 11 and flaps 50 mounted on both end portions of the main body 11.

The main body 11 is a tubular member that extends along a longitudinal axis X1 and has a tip end 12 and a base end 13. The tip end 12 is an end portion that is disposed on the liver side when it is placed in the bile duct. The base end 13 is an end portion that is disposed on the duodenal papilla side when it is placed in the bile duct.

In the following description, a direction in which the longitudinal axis X1 extends will be referred to as a “longitudinal direction A.” In addition, in the stent 10, the side on which the tip end 12 of the main body 11 is provided is referred to as the “tip end side (the distal side) A1” in the longitudinal direction A, and the side on which the base end 13 is provided is referred to as the “base end side (the proximal side) A2” in the longitudinal direction A.

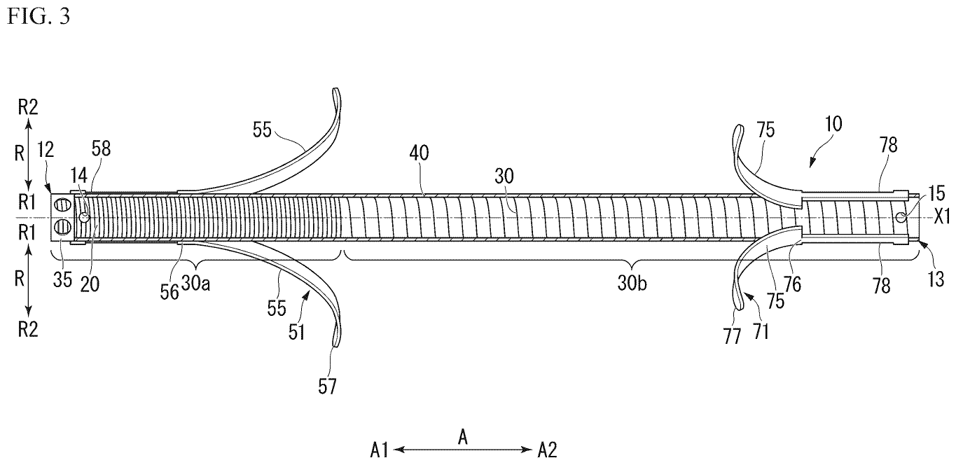

In addition, in the stent 10, a direction perpendicular to the longitudinal direction A is referred to as a “radial direction R,” the side approaching the longitudinal axis X1 is referred to as the “inner side R1” in the radial direction R, and the side away from the longitudinal axis X1 is referred to as the “outer side R2” in the radial direction R. FIG. 3 is a perspective view of the stent 10 showing an outer layer of the stent transparently.

The main body 11 includes an inner layer 20 made of a resin, a metal wire (a reinforcing material) 30 wound around the inner layer 20, and an outer layer 40 made of a resin that covers the inner layer 20 and the wire 30.

In the main body 11, the inner layer 20 and the outer layer 40 constitute a “tubular member” that extends in the longitudinal direction A.

The wire 30 is embedded in the main body 11 between an inner circumferential surface and an outer circumferential surface of the tubular member.

The main body 11 has, for example, the same outer diameter from the tip end 12 to the base end 13.

The inner layer 20 is a tube formed of a resin material, such as PTFE (polytetrafluoroethylene) or PFA (perfluoroalkoxylalkane), that has a smooth surface and is biocompatible.

The wire 30 is a coil-shaped member wound in a spiral shape around the longitudinal axis X1 on the outer circumferential surface of the inner layer 20. By winding the wire 30 in a spiral shape between the inner circumferential surface and the outer circumferential surface of the main body 11, buckling of the stent 10 can be curbed, and flexibility of the stent 10 can be improved.

The wire 30 is made of a material that is radiopaque, such as tungsten steel or stainless steel.

The main body 11 has a plurality of regions in which winding pitches of the wire 30 are different from one another. The plurality of regions in the main body 11 will be described in detail below.

The outer layer 40 is formed of a resin material having elasticity, flexibility and biocompatibility, such as urethane, polyethylene, or the like. The outer layer 40 is also provided in a gap between the wires 30 adjacent to each other in the longitudinal direction A.

The flap 50 has a first flap 51 provided on the tip end side A1 and a second flap 71 provided on the base end side A2.

The first flap 51 has a plurality of first locking members 55. The first locking members 55 are elongated members each of which has a first fixed end 56 and a first free end 57 and extends between the first fixed end 56 and the first free end 57.

In the first locking member 55, a rib (a first rib) 58 extends from the first fixed end 56 to the tip end side A1. In this embodiment, the width of the rib 58 is the same as the width of the first locking member 55.

The first locking member 55 is attached to the main body 11 by joining the rib 58 to the outer circumferential surface of the main body 11. The rib 58 is joined to the main body 11 by a joining method such as heat fusion or adhesion.

By joining the rib 58 to the main body 11, the first fixed end 56 of the first locking member 55 is fixed to the main body 11. In the first flap 51 connected to the main body 11, the first fixed end 56 is a boundary portion between a portion joined to the main body 11 by thermal welding, adhesion, or the like, and a portion not joined to the main body 11.

The first free end 57 is located on the outer side R2 with respect to the outer circumferential surface of the main body 11. The first locking member 55 extends from the first fixed end 56 toward the first free end 57, toward the base end side A2 and toward the outer side R2. The first locking member 55 gradually extends toward the outer side R2 and the base end side A2.

The second flap 71 has a plurality of second locking members 75. The second locking members 75 are elongated members each of which has a second fixed end 76 and a second free end 77 and extends between the second fixed end 76 and the second free end 77.

In the second locking member 75, a rib (a second rib) 78 extends from the second fixed end 76 to the base end side A2. In this embodiment, the width of the rib 78 is smaller than the width of the second locking member 75.

The second locking member 75 is attached to the main body 11 by joining the rib 78 to the outer circumferential surface of the main body 11. The rib 78 is joined to the main body 11 by a joining method such as heat fusion or adhesion.

By joining the rib 78 to the main body 11, the second fixed end 76 of the second locking member 75 is fixed to the main body 11. In the second flap 71 connected to the main body 11, the second fixed end 76 is a boundary portion between a portion joined to the main body 11 by thermal welding, adhesion, or the like, and a portion not joined to the main body 11.

The second free end 77 is located on the outer side R2 with respect to the outer circumferential surface of the main body 11. The second locking member 75 extends from the second fixed end 76 toward the second free end 77, toward the tip end side A1 and toward the outer side R2. The second locking member 75 gradually extends toward the outer side R2 and the tip end side A1.

In this embodiment, a length of the second locking member 75 in the longitudinal direction A is shorter than a length of the first locking member 55 in the longitudinal direction A.

The number of first locking members 55 in the first flap 51 and the number of second locking members 75 in the second flap 71 can be set as appropriate. In this embodiment, the first flap 51 has three first locking members 55, and the second flap 71 has three second locking members 75. The three first locking members 55 and the three second locking members 75 are attached to the outer circumferential surface of the main body 11 at equal intervals around the longitudinal axis X1.

In the first flap 51 and the second flap 71, phases on the outer circumferential surface of the main body 11 to which the first locking member 55 and the second locking member 75 are attached may be the same or different. In the first flap 51 and the second flap 71, the number of first locking members 55 and the number of second locking members 75 may be the same or different.

The main body 11 has a first region 30a and a second region 30b in which a winding pitch of the wire 30 is different from each other. The winding pitch of the wire 30 in the first region 30a is generally uniform, and the winding pitch of the wire 30 in the second region 30b is generally uniform.

In the longitudinal direction A, a tip end of the first region 30a is located at the tip end 12 of the main body 11. The second region 30b is adjacent to the first region 30a in the longitudinal direction A and is provided on the base end side A2 of the first region 30a.

The second region 30b is a region in which the number of turns of the wire 30 per unit length is smaller than that in the first region 30a. That is, the second region 30b is a region in which the wire 30 is wound in a spiral shape with a larger winding pitch than that in the first region 30a.

Therefore, in the main body 11, the first region 30a has a greater rigidity than the second region 30b, and is less likely to deform due to a force applied in the longitudinal direction A or the radial direction R than the second region 30b.

Regarding a winding angle (an inclination) of the wire 30 with respect to the longitudinal axis X1, the winding angle of the wire 30 in the first region 30a is closer to a right angle than the winding angle of the wire 30 in the second region 30b.

A length of the second region 30b is longer than a length of the first region 30a in the longitudinal direction A. In addition, in the longitudinal direction A, a distance from a tip end of the first region 30a to the first fixed end 56 is shorter than a distance from the first fixed end 56 to a base end of the first region 30a.

A boundary between the first region 30a and the second region 30b is provided at the same position as the first free end 57 in the longitudinal direction A. The boundary between the first region 30a and the second region 30b does not have to be at the same position as the first free end 57 strictly.

By arranging the boundary between the first region 30a and the second region 30b at a position corresponding to the first free end 57, for example, when the stent 10 is placed in a bile duct, the position of the first free end 57 can be confirmed by visually observing the boundary between the first region 30a and the second region 30b under X-ray fluoroscopy.

The base end of the second region 30b is located at the base end 13 of the main body 11. The wire 30 is provided from the tip end 12 to the base end 13 of the main body 11.

The wire 30 is fixed to the main body 11 by a metal ring 35. The ring 35 is an annular member of which a central axis is the longitudinal axis X1, and is provided at the tip end of the first region 30a.

A first through hole (a through hole) 14 and a second through hole (a through hole) 15 that pass through the main body 11 from the inner circumferential surface to the outer circumferential surface are formed in the main body 11.

In the stent 10 placed in the bile duct, bile can flow from the outer circumferential surface side of the main body 11 into an internal space of the main body 11 via the through holes 14 and 15. The number and positions of the through holes 14 and 15 can be set appropriately. A part or the whole of the through holes 14 and 15 may overlap the first rib 58 or the second rib 78.

In this embodiment, the first through hole 14 is provided on the tip end side A1 of the main body 11, and is provided at a position at which it overlaps the first region 30a in the longitudinal direction A. Specifically, the first through hole 14 is provided on the tip end side A1 with respect to the first fixed end 56.

The second through hole 15 is provided on the base end side A2 of the main body 11, and is provided at a position at which it overlaps the second region 30b in the longitudinal direction A.

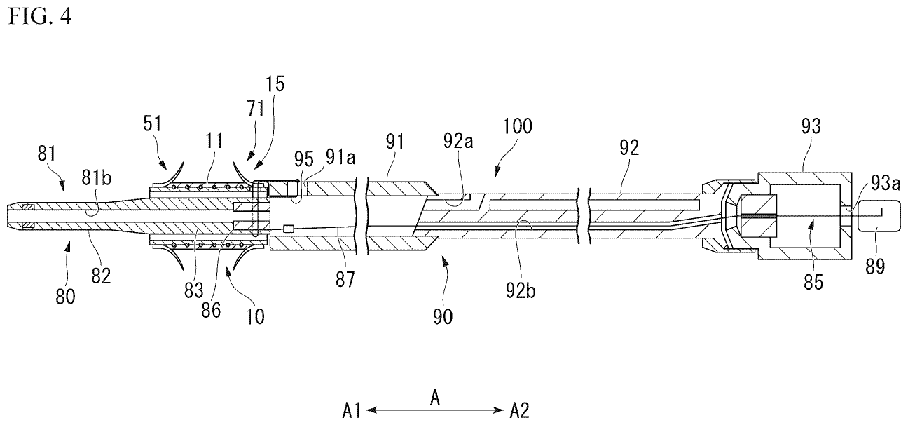

FIG. 4 is a schematic cross-sectional view showing a structure of the stent delivery device 1.

The delivery catheter 100 includes a guide catheter 80 and a pusher catheter 90.

The guide catheter 80 has a tube (a guide tube) 81 through which a guide wire can be inserted, and a traction part 85 for traction of the tube 81 toward the base end side A2. The guide wire is an elongated member used when the stent 10 is placed in the bile duct.

The tube 81 is a tubular member made of a resin, and has an inner cavity through which the guide wire can be inserted. The tube 81 is flexible enough to be deformed when the tube 81 comes into contact with biological tissue during use of the stent delivery device 1.

The tube 81 is an elastic member having a restoring force, and when no external force is applied, the tube 81 is straightened by the restoring force. An inner circumferential surface 81b of the tube 81 is circular when seen in a cross section of the tube 81 in the radial direction.

The tube 81 has a small diameter portion 82 located on the tip end side A1 and a large diameter portion 83 located on the base end side A2. The outer diameter of the large diameter portion 83 is larger than the outer diameter of the small diameter portion 82.

An outer circumferential surface of the small diameter portion 82 and an outer circumferential surface of the large diameter portion 83 are connected by a smoothly curved surface. As a result, an outer diameter of the tube 81 gradually increases from the small diameter portion 82 toward the large diameter portion 83.

The outer diameters of the small diameter portion 82 and the large diameter portion 83 are smaller than the inner diameter of the stent 10. Therefore, the stent 10 can be mounted in the tube 81 by inserting the tube 81 into the stent 10.

In cases in which the diameter of the stent 10 is small, or the like, the outer diameter of the tube 81 may be constant in the longitudinal direction A.

The traction part 85 has a pipe 86, a wire 87, and an operating part 89. The pipe 86 is a metallic tubular member that is open at both ends in the axial direction.

The pipe 86 is coaxial with the tube 81 and is embedded in a wall of the tube 81. The pipe 86 is provided at a base end portion of the large diameter portion 83.

The wire 87 is an elongated member made of a metal, a tip end portion thereof is welded to the pipe 86, and a base end portion thereof is connected to the operating part 89.

The pusher catheter 90 has a single lumen tube (a second tube) 91, a multi-lumen tube (a first tube) 92, and a gripping portion 93.

The single lumen tube 91 is a tubular member having an internal space (a second lumen) into which the large diameter portion 83 of the tube 81 can advance. The single lumen tube 91 is flexible.

A surface (a tip end surface) of the single lumen tube 91 on the tip end side A1 is a plane perpendicular to a central axis of the single lumen tube 91. The tip end surface of the single lumen tube 91 can come into contact with the base end of the stent 10 to support the stent 10.

A thickness of the single lumen tube 91 is greater than a thickness of the main body 11 of the stent 10. The thickness of the single lumen tube 91 indicates a difference in dimension between the outer diameter and the inner diameter of the single lumen tube 91. The thickness of the main body 11 indicates a difference in dimension between the outer diameter and the inner diameter of the main body 11.

The single lumen tube 91 has a length that allows the large diameter portion 83 of the tube 81 to be completely accommodated inside the single lumen tube 91. The material of the single lumen tube 91 is not particularly limited, but a resin is preferable.

The multi-lumen tube 92 is fixed to a base end portion of the single lumen tube 91. The multi-lumen tube 92 has a guide wire lumen 92a through which a guide wire is inserted, and a wire lumen (a first lumen) 92b.

The guide wire lumen 92a opens at a tip end of the multi-lumen tube 92, and opens at a side surface of the multi-lumen tube 92 on the base end side A2 with respect to the tip end of the multi-lumen tube 92.

The wire 87 of the guide catheter 80 is inserted through the wire lumen 92b. The wire lumen 92b opens at the tip end and the base end of the multi-lumen tube 92.

The gripping portion 93 is connected to the base end of the multi-lumen tube 92. The gripping portion 93 is a generally columnar member having a diameter larger than the multi-lumen tube 92. An outer circumferential surface of the gripping portion 93 may be formed with protrusions and recesses for anti-slip purposes or the like.

A through hole (a gripping through hole) 93a that communicates with the wire lumen 92b is formed in the gripping portion 93. In this embodiment, the through hole 93a is located on an extension line of the central axis of the multi-lumen tube 92 toward the base end side A2.

The wire 87 of the guide catheter 80 is inserted through the through hole 93a. The wire 87 and the operating part 89 connected to the base end of the wire 87 extend toward the base end side A2 with respect to the through hole 93a.

A hole 91a that communicates with the internal space (the second lumen) of the single lumen tube 91 is provided in the outer circumferential surface at the tip end portion of the single lumen tube 91.

A thread (connecting member) 95 is passed through the hole 91a. The thread 95 is formed to have a loop (annular) shape in which end portions thereof are connected to each other. The looped thread 95 passes through the second through hole 15 from the outer side R2 to the inner side R1 in the main body 11 of the stent 10, and advances into the inside of the main body 11.

The tube 81 of the guide catheter 80 is inserted in the longitudinal direction A through the loop of the thread 95 inside the stent 10.

Next, an operation of the stent delivery device 1 when it is used will be described.

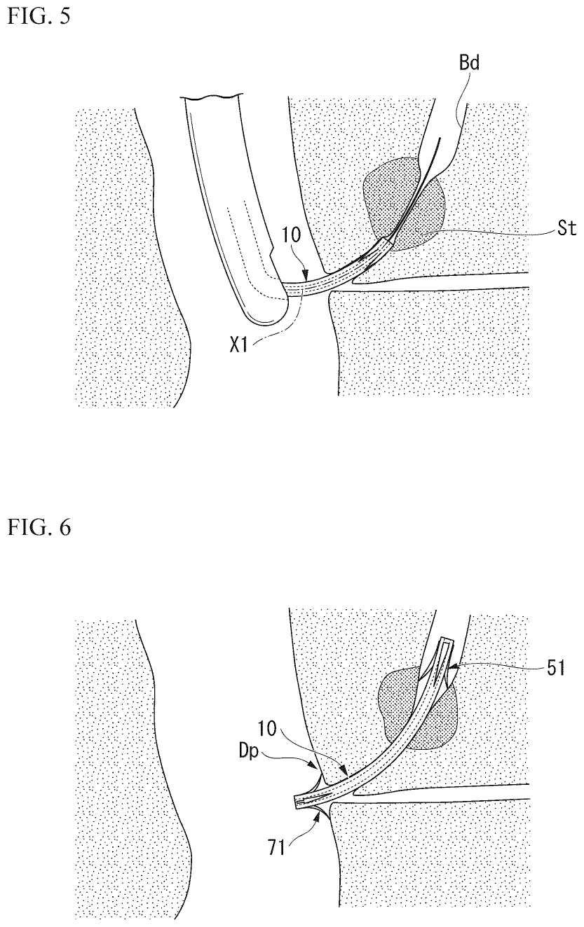

FIG. 5 is a diagram showing a process of placing the stent 10 by the stent delivery device 1. FIG. 6 is a diagram showing the placed stent 10.

First, a surgeon inserts a base end portion of the guide wire protruding from a forceps port of an endoscope into a tip end opening of the tube 81 of the stent delivery device 1 in which the stent 10 is mounted.

The guide wire advances from a base end opening of the tube 81 into the second lumen of the single lumen tube 91. The surgeon advances the base end portion of the guide wire into the guide wire lumen 92a, and causes the guide wire to protrude from a base end side opening of the guide wire lumen 92a.

The surgeon inserts the stent delivery device 1 with a guide wire passing therethrough into a channel of the endoscope, and causes the tip end portion of the stent delivery device 1 to protrude from a tip end of the channel. The surgeon operates a hoisting table of the endoscope to advance the stent delivery device 1 into the bile duct while pointing the tip end of the stent delivery device 1 toward the duodenal papilla along the guide wire.

As shown in FIG. 5, the stent delivery device 1 advances into a bile duct Bd while expanding a stenosis portion St in the bile duct Bd.

At this time, the stent 10 mounted in the stent delivery device 1 advances into the bile duct Bd from the tip end side A1. As described above, in the stent 10, the first region 30a with a rigidity greater than the second region 30b disposed on the base end side A2 is disposed on the tip end side A1.

Therefore, the stent 10 can advance into the bile duct Bd while expanding the stenosis portion St and sufficiently maintaining a space within the bile duct Bd.

The surgeon advances the stent 10 into the bile duct Bd to a position at which the first flap 51 is disposed inward (on the tip end side A1) with respect to the stenosis portion St, and then moves the stent delivery device 1 back and forth to determine the placement position of the stent 10.

In the stent delivery device 1, as long as the tube 81 is inserted through the loop of the thread 95, the stent 10 will not come off the pusher catheter 90, and thus the stent 10 can be pulled back by retracting the stent delivery device 1. Thus, the surgeon can easily adjust the position of the stent 10.

After the placement position of the stent 10 is determined, the surgeon pulls the operating part 89 toward the hand side (the base end side A2) while holding the pusher catheter 90. By pulling the operating part 89 toward the hand side, the wire 87 and the tube 81 are retracted. At this time, the stent 10 does not retract because it is in contact with the pusher catheter 90 in the longitudinal direction A.

When the tube 81 is retracted and comes out of the stent 10 and the thread 95, engagement between the stent 10 and the pusher catheter 90 by the thread 95 is released, and the stent 10 is placed at a predetermined position in the bile duct Bd, as shown in FIG. 6.

After the stent 10 is placed, as shown in FIG. 6, the first flap 51 is disposed inside the stenosis portion St, the second flap 71 is disposed near the duodenal papilla Dp outside the bile duct Bd, and thus the placement position of the stent 10 is appropriately maintained.

In the stent 10 placed in the bile duct Bd, the second region 30b disposed on the base end side A2 has a lower rigidity than the first region 30a and a longer length in the longitudinal direction A than the first region 30a.

Therefore, the stent 10 is placed in the bile duct Bd in a state in which it sufficiently conforms to a shape of the bile duct Bd.

Next, an operation of retrieving the stent 10 placed in the bile duct Bd through the channel of the endoscope will be described.

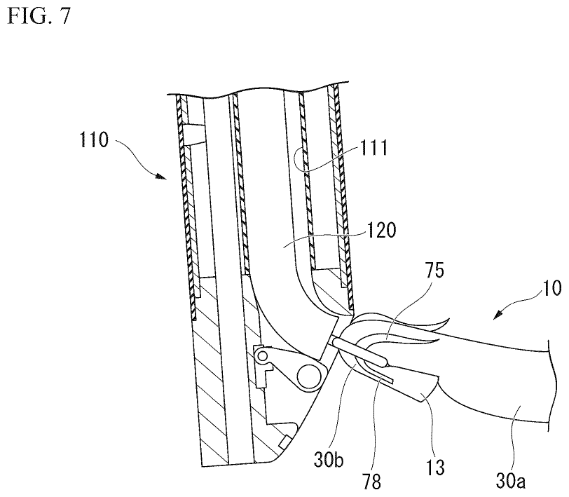

FIG. 7 is a diagram showing a process of drawing the stent 10 into the channel of the endoscope.

The surgeon introduces a side view type endoscope to the vicinity of the duodenal papilla Dp, protrudes a retrieval tool such as grasping forceps or a snare from the channel, and holds the base end portion of the stent 10 with the retrieval tool.

Next, the surgeon retracts the retrieval tool 120 that holds the stent 10. As shown in FIG. 7, when the stent 10 reaches a channel tip end opening of the side view type endoscope (the endoscope) 110, the stent 10 is drawn into a channel 111 of the endoscope 110 while being folded back at a portion held by the retrieval tool 120.

In the stent 10, the portion held by the retrieval tool 120 and the portion folded back are the second region 30b disposed on the base end side A2. Since the second region 30b which has a lower rigidity than the first region 30a is folded back, the surgeon can easily collapse the stent 10 and draw it into the channel 111.

In the stent 10, a length of the second region 30b is longer than a length of the first region 30a in the longitudinal direction A. Therefore, when the stent 10 is retrieved with the retrieval tool 120, the surgeon can reliably fold back and collapse the second region 30b.

Also, as described above, the width of the second rib 78 is smaller than the width of the second locking member 75. Therefore, a volume per unit length of the second rib 78 is smaller than a volume per unit length of the second locking member 75. Thus, it is possible to curb the second rib 78 hindering the second region 30b from being collapsed.

The medical stent (the stent) 10 of this embodiment includes a main body 11 having a tubular member (an inner layer 20 and an outer layer 40) that extends in a longitudinal direction A, and a reinforcing material (a wire) 30 wound in a spiral shape around a longitudinal axis X1 extending in the longitudinal direction A and extends in the longitudinal direction A, extending in the longitudinal direction A, and embedded between an inner circumferential surface of the tubular member and an outer circumferential surface of the tubular member. The main body 11 has a first region 30a and a second region 30b in which winding pitches of the reinforcing material 30 are different from each other.

The second region 30b is a region in which the reinforcing material 30 is wound in a spiral shape at a winding pitch greater than that in the first region 30a, and is longer in the longitudinal direction A than the first region 30a.

The stent delivery device 1 of this embodiment includes a guide catheter 80 having a guide tube (tube) 81 through which a guide wire can be inserted and a wire 87 connected to the guide tube 81, a pusher catheter 90 having a first tube (a multi-lumen tube) 92 having a first lumen (a wire lumen) 92b through which the wire 87 can be inserted and a second tube (a single lumen tube) 91 having a second lumen that is connected to a tip end portion of the first lumen 92b and through which the guide tube 81 can be inserted, a connecting member (a thread) 95 having a loop through which the guide tube 81 can be inserted and connected to a tip end portion of the pusher catheter 90, and a stent 10 located on the tip end side A1 with respect to a tip end of the second tube 91.

The guide tube 81 is inserted through the tubular member of the stent 10.

The loop of the connecting member 95 passes through the tubular member of the stent 10 from the inner circumferential surface to the outer circumferential surface, and is inserted through a through hole (a second through hole) 15 provided on the base end side A2.

The stent 10 is positioned with respect to the pusher catheter 90 by inserting the guide tube 81 through the loop of the connecting member 95.

Therefore, according to the medical stent 10 and the stent delivery device 1 of this embodiment, it is possible to provide a medical stent 10 and a stent delivery device 1 that can be placed in the bile duct Bd in a state in which they sufficiently conform to the shape of the bile duct Bd.

### Second Embodiment

A medical stent (stent) 10A according to a second embodiment of the present invention will be described with reference to the drawings.

In the following description, components common to those already described will be given the same reference numerals, and duplicated description will be omitted.

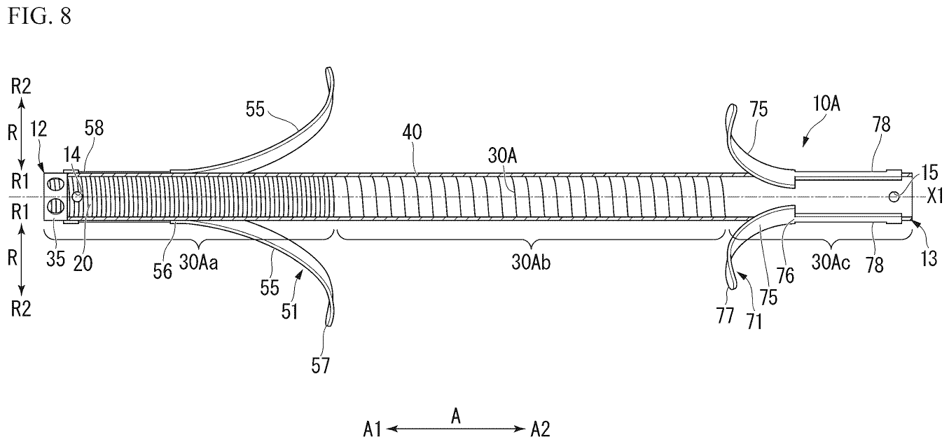

FIG. 8 is a perspective view of the stent 10A according to the second embodiment.

In the stent 10A, a first region 30Aa, a second region 30Ab, and a third region 30Ac are provided in this order from the tip end side A1.

The first region 30Aa and the second region 30Ab are regions in which a wire 30A is provided. The wire 30A is a coil-shaped member wound in a spiral shape on the outer circumferential surface of the inner layer 20, similar to the wire 30 of the first embodiment.

The first region 30Aa and the second region 30Ab are regions in which the wire 30A is wound with different winding pitches.

A winding pitch of the wire 30A in the first region 30Aa in this embodiment is similar to the winding pitch of the wire 30 in the first region 30a in the first embodiment.

Moreover, a winding pitch of the wire 30A in the second region 30Ab in this embodiment is similar to the winding pitch of the wire 30 in the second region 30b in the first embodiment.

That is, the second region 30Ab of this embodiment is a region in which the wire 30A is wound at a larger winding pitch than in the first region 30Aa.

A length of the second region 30Ab is longer than a length of the first region 30Aa in the longitudinal direction A. In addition, a base end of the second region 30Ab in this embodiment is located on the tip end side A1 of the base end 13 of the main body 11 in the longitudinal direction A.

Specifically, the base end of the second region 30Ab is disposed at the same position as the second free end 77 in the longitudinal direction A. The base end of the second region 30Ab does not have to be at the same position as the second free end 77 strictly.

The third region 30Ac is a region in which the wire 30A is not provided. That is, in the stent 10A of this embodiment, the wire 30A is not provided in a region on the base end side A2 with respect to the second free end 77.

Therefore, a rigidity of the stent 10A is greatest in the first region 30Aa and smallest in the third region 30Ac. A rigidity of the second region 30Ab is less than that of the first region 30Aa and greater than that of the third region 30Ac.

When the stent 10A advances into the stenosis portion St of the bile duct Bd, a force compressing the stent 10A in the longitudinal direction A acts on the stent 10A.

Because the stent 10A has the first region 30Aa, the second region 30Ab and the third region 30Ac, the above-described force acts in a dispersed manner at a boundary portion between the first region 30Aa and the second region 30Ab, and at a boundary portion between the second region 30Ab and the third region 30Ac. As a result, buckling of the stent 10A during placement of the stent 10A can be curbed.

In the stent 10A, the second locking member 75 and the rib 78 are provided in the third region 30Ac, and thus the second locking member 75 and the rib 78 can provide a predetermined rigidity.

Therefore, in the stent 10A, the rigidity of each of the first region 30Aa, the second region 30Ab, and the third region 30Ac can be gradually changed.

The medical stent 10A according to this embodiment can be placed within the bile duct Bd in a state in which it sufficiently conforms to the shape of the bile duct Bd, and further, buckling of the stent 10A during the placement of the stent 10A can be suitably curbed.

### Third Embodiment

A medical stent (a stent) 10B according to a third embodiment of the present invention will be described with reference to the drawings.

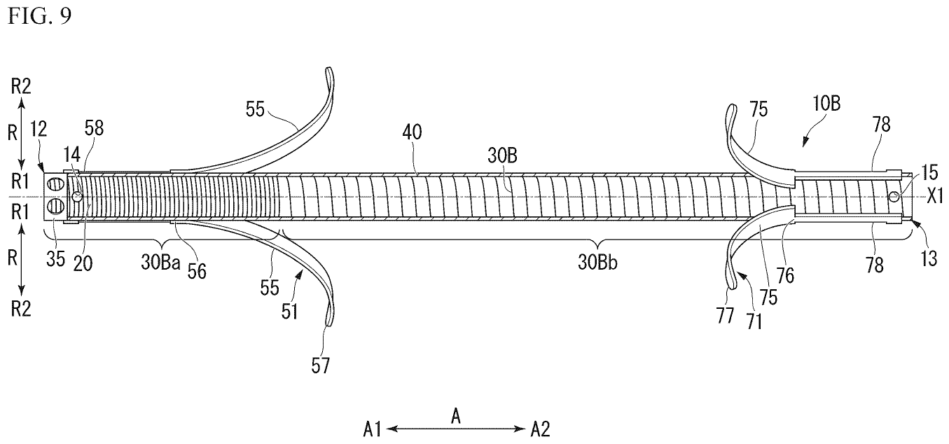

FIG. 9 is a perspective view of the stent 10B according to the third embodiment.

In the stent 10B, a first region 30Ba and a second region 30Bb are provided in this order from the tip end side A1.

The first region 30Ba and the second region 30Bb are regions in which a wire 30B is provided. The wire 30B is a coil-shaped member wound in a spiral shape on the outer circumferential surface of the inner layer 20, similar to the wire 30 of the first embodiment.

The first region 30Ba and the second region 30Bb are regions in which the wire 30B is wound with different winding pitches.

The winding pitch of the first region 30Ba in this embodiment is similar to the winding pitch of the wire 30 in the first region 30a in the first embodiment.

The winding pitch of the second region 30Bb in this embodiment is similar to the winding pitch of the wire 30 in the second region 30b in the first embodiment. That is, the second region 30Bb of this embodiment is a region in which the wire 30B is wound at a larger winding pitch than the first region 30Ba.

In the longitudinal direction A, a length of the second region 30Bb is longer than a length of the first region 30Ba, and a base end of the second region 30Bb is located at the base end 13 of the main body 11.

In the stent 10B of this embodiment, a boundary between the first region 30Ba and the second region 30Bb is provided at a position at which it overlaps the first locking member 55 in the longitudinal direction A.

By arranging the boundary between the first region 30Ba and the second region 30Bb at a position corresponding to the first locking member 55, occurrence of kinking (bending, collapsing, or the like) at the boundary between the first region 30Ba and the second region 30Bb in the stent 10B can be curbed when the stent 10B advances into the bile duct Bd.

The medical stent 10B of this embodiment can be placed within the bile duct Bd in a state in which it sufficiently conforms to the shape of the bile duct Bd, and further, can effectively curb the occurrence of the kinking at the boundary between the first region 30Ba and the second region 30Bb when the stent 10B is placed.

Although each of the embodiments of the present invention has been described in detail above with reference to the drawings, the specific configuration is not limited to these embodiments, and design modifications and the like that do not deviate from the gist of the present invention are also included. Furthermore, the components shown in the above-described embodiments and the modified examples shown below can be appropriately combined to form a configuration.

### Modified Example 1

In each of the above embodiments, the main body 11 has the same outer diameter from the tip end 12 to the base end 13, but the aspect of the main body 11 is not limited thereto.

For example, the main body of the stent may have a reduced diameter portion, in which a diameter of the outer circumferential surface is reduced toward the tip end side A1, at the tip end portion.

The reduced diameter portion is provided, for example, on the tip end side A1 with respect to the first fixed end 56. In this reduced diameter portion, the diameter of the inner circumferential surface of the main body of the stent may be reduced toward the tip end side A1, or a diameter of the wire (the reinforcing material) embedded in the main body may be reduced toward the tip end side A1.

Due to the tip end of the stent being tapered by the reduced diameter portion, when the stent advances into the bile duct Bd, the tip end of the stent can easily advance into the stenosis portion St.

What is claimed is:

1. A medical stent comprising:
a main body including a tubular member configured to extend in a longitudinal direction, and a reinforcing material wound in a spiral shape around a longitudinal axis extending in the longitudinal direction and embedded between an inner circumferential surface of the tubular member and an outer circumferential surface of the tubular member,
wherein the main body has a first region and a second region in which winding pitches of the reinforcing material are different from each other, and
the second region is a region in which the reinforcing material is wound in a spiral shape at a winding pitch larger than that of the first region, and is longer than the first region in the longitudinal direction.

2. The medical stent according to claim 1, further comprising:
a first locking member having a first fixed end fixed to a tip end side of the main body in the longitudinal direction and a first free end located on an outer side of the main body in a radial direction with respect to the outer circumferential surface of the tubular member, the first locking member extending from the first fixed end to the first free end toward a base end side in the longitudinal direction; and
a second locking member having a second fixed end fixed to the base end side of the main body and a second free end located on the outer side with respect to the outer circumferential surface of the tubular member, the second locking member extending from the second fixed end to the second free end toward the tip end side.

3. The medical stent according to claim 2, wherein the first region is provided on the tip end side with respect to the second region,
a tip end of the first region is located at a tip end of the main body, and
in the longitudinal direction, a distance from the tip end of the first region to the first fixed end is shorter than a distance from the first fixed end to a base end of the first region.

4. The medical stent according to claim 2, wherein a boundary between the first region and the second region is provided at approximately the same position as the first free end in the longitudinal direction.

5. The medical stent according to claim 2, wherein a boundary between the first region and the second region is provided at a position at which it overlaps the first locking member in the longitudinal direction.

6. The medical stent according to claim 1, wherein a base end of the second region is located at a base end of the main body.

7. The medical stent according to claim 1, wherein a base end of the second region is located on a tip end side in the longitudinal direction with respect to a base end of the main body.

8. The medical stent according to claim 2, wherein a base end of the second region is located at approximately the same position as the second free end in the longitudinal direction.

9. The medical stent according to claim 1, wherein the main body has a through hole that passes therethrough from the inner circumferential surface to the outer circumferential surface, and
the through hole is provided to overlap the first region in the longitudinal direction.

10. The medical stent according to claim 1, further comprising an annular ring having a central axis on the longitudinal axis,
wherein the ring is provided at a tip end of the first region and fixes the reinforcing material to the main body.

11. The medical stent according to claim 1, wherein the main body has a reduced diameter portion, in which a diameter of the outer circumferential surface of the main body is reduced toward a tip end side in the longitudinal direction, at a tip end.

12. A stent delivery device comprising:
a guide catheter including a guide tube through which a guide wire is configured to be inserted and a wire connected to the guide tube;
a pusher catheter including a first tube having a first lumen through which the wire is configured to be inserted, and a second tube having a second lumen connected to a tip end portion of the first lumen and through which the guide tube is configured to be inserted;
a connecting member having a loop through which the guide tube is configured to be inserted, and connected to a tip end portion of the pusher catheter; and
a stent including a main body having a tubular member configured to extend in a longitudinal direction and through which the guide tube is inserted, a reinforcing material embedded between an inner circumferential surface of the tubular member and an outer circumferential surface of the tubular member, and a through hole configured to pass through the tubular member from the inner circumferential surface to the outer circumferential surface and provided on a base end side in the longitudinal direction, the stent being located on a tip end side in the longitudinal direction with respect to a tip end of the second tube,
wherein the reinforcing material is wound in a spiral shape around a longitudinal axis extending in the longitudinal direction and extends in the longitudinal direction,
the main body has a first region and a second region in which winding pitches of the reinforcing material are different from each other,
the second region is a region in which the reinforcing material is wound in a spiral shape at a winding pitch larger than that in the first region, and the second region is provided on the base end side with respect to the first region and is longer than the first region in the longitudinal direction,
the loop of the connecting member is inserted through the through hole, and
the stent is positioned with respect to the pusher catheter by inserting the guide tube through the loop.
